(19) United States
(12) Patent Application Publication
(10) Pub. No.: US 2023/0310480 A1
(43) Pub. Date: Oct. 5, 2023

(54) PHARMACEUTICAL COMBINATION THERAPY AND PREVENTION WITH APROTININ + MOLNUPIRAVIR OF SARS-CoV-2 AND/OR DISEASE ASSOCIATED WITH THIS INFECTION, INCLUDI COVID-19

(71) Applicants: Alexandre Vasilievich Ivachtchenko, Hallandale, FL (US); Alena Alexandrovna Ivachtchenko, Hallandale, FL (US); Nikolay Filippovich Savchuk, Rancho Santa Fe, CA (US); ASAVI LLC, Hallandale, FL (US)

(72) Inventors: Alexandre Vasilievich Ivachtchenko, Hallandale, FL (US); Andrey Alexandrovich Ivashchenko, Moscow (RU); Alena Alexandrovna Ivachtchenko, Hallandale, FL (US); Ilya Andreevich Ivashchenko, Moscow region (RU); Nikolay Filippovich Savchuk, Rancho Santa Fe, CA (US)

(73) Assignees: ALEXANDRE VASILIEVICH IVACHTCHENKO, Hallandale, FL (US); ALENA ALEXANDROVNA IVACHTCHENKO, Hallandale, FL (US); NIKOLAY FILIPPOVICH SAVCHUK, Rancho Santa Fe, CA (US); ASAVI LLC, Hallandale, FL (US)

(21) Appl. No.: 17/709,608

(22) Filed: Mar. 31, 2022

(51) Int. Cl. A61K 31/7068; A61K 38/57; A61K 45/06

(52) U.S. Cl. CPC A61K 31/7068; A61K 38/57; A61K 45/06

(57) ABSTRACT

The application relates to pharmaceutical combinations, methods, and therapies for the treatment and prevention of SARS-CoV-2 infection and related diseases. The combinations contain aprotinin and molnupiravir, and/or favipiravir. The application also includes related pharmaceutical kits. Further details are described in the specification.

### FIELD OF INVENTION

The application relates to pharmaceutical combinations, methods, and therapies for the treatment and prevention of SARS-CoV-2 infection and related diseases. The combinations contain aprotinin and molnupiravir, and/or favipiravir. The application also includes related pharmaceutical kits. Further details are described in the specification.

### BACKGROUND

SARS diseases (e.g., COVID-19) are caused by coronaviruses. The SARS viruses have a tropism for the epithelium of the mucous membranes of the respiratory system. They are characterized by catarrhal damage to the mucous membranes of the larynx, trachea, and bronchi with involvement of the lungs in the process. The infections are transmitted mainly by aerosol transmission.

Severe Acute Respiratory Syndrome Coronavirus 2 (SARS-CoV-2) is RNA virus that causes a 2019 Coronavirus Disease (COVID-19). SARS-CoV-2 is responsible for the ongoing COVID-19 pandemic. SARS-CoV-2 is a virus that belongs to a type of coronavirus associated with SARS-CoV (Coronaviridae Study Group of the International Committee on Taxonomy of Viruses (April 2020). “The species Severe acute respiratory syndrome—related coronavirus: classifying 2019-nCoV and naming it SARS-CoV-2”. Nat. Microbiol. 2020, 5(4), 536-544. doi:10.1038/s41564-020-0695-z). This virus was first identified in December 2019 in Wuhan city, Hubei province, China. On Mar. 11, 2020, WHO declared the outbreak a public health emergency of international concern. SARS-CoV-2 is the successor to the SARS-CoV-1 virus that caused the SARS outbreak in 2002-2004 (“New coronavirus stable for hours on surfaces”. National Institutes of Health (NIH). NIH.gov. 17 Mar. 2020. Archived from the original on 23 Mar. 2020. Retrieved 4 May 2020). SARS-CoV-2 has undergone many changes in two years, and each new mutation has been more perfect than the previous one. First discovered in India in December 2020, the Delta mutation is spreading across continents at an alarming rate. Delta penetrates lung cells more easily than the original virus (the virus that circulated in the early stages of the pandemic). In addition, the Delta strain is more effective in combining infected lung cells with uninfected ones. This could contribute to the more severe course of COVID-19. It is currently the predominant variant of SARS-CoV-2 worldwide. Delta is believed to be more than twice as infectious as previous SARS-CoV-2 variants (K. Katella. 5 Things to Know About the Delta Variant. Yale Medicine Nov. 19, 2021.

The new variant of coronavirus Omicron was detected in laboratories in Botswana and South Africa on 22 Nov. 2021. The variant has an unusually large number of mutations, several of which are novel and a significant number of which affect the spike protein targeted by most COVID-19 vaccines at the time of discovering the Omicron variant. This level of variation has led to concerns regarding its transmissibility, immune system evasion, and vaccine resistance. Omicron spreads faster than any previously known variant.

As of Dec. 17, 2021, 77 countries have now reported cases of Omicron, and “the reality is that Omicron is probably in most countries, even if it hasn't been detected yet.” (L. Smith-Spark, What can the world learn from countries where Omicron is surging, CNN Fri Dec. 17, 2021.

As of Jan. 28, 2022, 364,191,494 confirmed cases of people infected with coronavirus were registered in the world, of which 5,631,457 people have unfortunately died.

Vaccination remains one of the main public health interventions to combat SARS-CoV-2. However, vaccine development times of at least six months limit their applicability during outbreaks of new strains of SARS-CoV-2, like the Omicron strain. Therefore, developing highly effective anticoronavirus drug remains an urgent task.

Prior attempts have been made to develop an effective anticoronavirus drug, for example, a pharmaceutical combination therapy of molnupiravir (MPV) and favipiravir (FPV) against SARS-CoV-2 infection. These combination of these two oral drugs (for which there is evidence that they exert antiviral activity in COVID-19 patients) is effective in the treatment of SARS-CoV-2 infections in hamsters and largely reduces transmission of the virus to uninfected contact sentinels. This combination demonstrates synergistic antiviral activity, and leads to a strong reduction in both total viral load and infectious virus when treatment is administered before or very rapidly after infection. (R. Abdelnabi et al. The combined treatment of Molnupiravir and Favipiravir results in a potentiation of antiviral efficacy in a SARS-CoV-2 hamster infection model. EBioMedicine 2021, 72, 103595, doi: 10.1016/j.ebiom.2021.103595)

The method of combination therapy discussed above, however, is only intended to treat a mild or early SARS-CoV-2 infection and does not provide the treatment of the COVID-19 patients with moderate and severe disease.

A further example provides a pharmaceutical combination therapy for patients with moderate COVID-19 infection by administration of intravenous aprotinin (APR) and oral avifavir (favipiravir, FPV) combination therapy. This therapy is more effective because primary and secondary efficacy endpoints of therapy by the APR+FPV combination are significantly better than efficacy endpoints of therapy by the individual components (Table 1). (Ivashchenko A. A. et al., Effect of Aprotinin and Avifavir® Combination Therapy for Moderate COVID-19 Patients. Viruses 2021, 13, 1253, doi: 10.3390/v13071253.).

TABLE 1
Primary and secondary efficacy endpoints of therapy by the intravenous APR,
oral FPV and their combination.

| Primary and Secondary Efficacy Endpoints | APR + SOC | FPV + SOC) | APR + FPV + SOC |
|---|---|---|---|
| Median time to elimination of SARS-CoV- | 7.5 (6-9) | 4.5 (4-9) | 3.5 (3-4) |
| 2 confirmed by RT-PCR, days (IQR) | | | |
| Median time to normalization of CRP | 6.0 (6-6) | 14.0 (5.5-14) | 3.5 (3-5) |
| concentration (≤10 mg/L) in patient's | | | |
| blood, days (IQR) | | | |
| Median time to normalization of D-dimer | 4.5 (3-6) | NA | 5.0 (4-5) |
| concentration (<253 ng/ml) in patient's | | | |
| blood, days (IQR) | | | |
| Median time to normalization of body | 3.0 (2-3) | 2.0 (1-3) | 1.0 (1-3) |
| temperature (<37° C.), days (IQR) | | | |
| Median time to improvement in clinical | 11.0 (6-11) | 14.0 (11.5-16) | 5.0 (5-5) |
| status by 2 points on the WHO-OSCI, days | | | |
| (IQR) | | | |

However, the foregoing treatments have the disadvantage of requiring two drugs with different routes of administration, i.e. oral and intravenous.

### TERMS USED IN THE DESCRIPTION

The term “drug” (also called medicine, medicament, pharmaceutical composition, or medicinal drug) refers to a drug used to diagnose, cure, treat, or prevent disease and means a substance (or a mixture of substances in the form of a pharmaceutical composition).

The term “oral drug” refers to solutions, powders, tablets, capsules, and pills that are taken by mouth and swallowed.

The term “parenteral drug” refers to drugs that are injected into the body bypassing the gastrointestinal tract. Parenteral drugs are solutions for injection, inhalation, sprays, including for nasal or drip application, and other finished dosage forms, in this case intended for the treatment and prevention of viral infections and diseases caused by them.

The term “parenteral therapies” are administration of drugs is primarily injections (intravenously, into the muscles, under the skin), inhalations and nasally (spray, drops).

The term “pharmaceutical composition” as used herein means a composition comprising at least two active ingredients (substances), namely aprotinin an inhibitor of RNA viruses, and at least one excipient.

The term “parenteral pharmaceutical composition (PPC)” is intended for parenteral administration of drugs into the body of a patient. These are primarily intravenous, inhalation and nasal routes of drug administration.

The term “excipient” as used herein refers to a compound that is used to prepare a pharmaceutical composition and is generally safe, non-toxic, and neither biologically nor otherwise undesirable, and includes excipients that are acceptable to humans and animals. This invention uses primarily excipients selected from the series: water, sodium chloride, L-lysine monohydrate, 2-hydroxy-beta-cyclodextrin, betadex sulfobutyl ether sodium, sodium hydroxide, hydrochloric acid, benzyl alcohol, ethanol, glycerin, dimethyl sulfoxide, peppermint oil, 1,1,1,2-tetrafluoroethane, and some others.

The term “pharmaceutical kit” as used herein means a kit including at least two drugs: Lagevrio® or molnupiravir saline solution, or its lyophilizate and the parenteral drug Trasylol®, or Gordox®, or Aprotex®, or Antagosan®, or Contrycal®, or Traskolan®, or others parenteral drugs including aprotinin, or aqueous or saline solution containing aprotinin.

The term “pharmaceutical combination therapy” is therapy that uses at least two drugs. “Pharmaceutical combination therapy” may be achieved by prescribing/administering separate drugs, or dosage forms that contain at least two active ingredients (such as fixed-dose combinations).

The term “therapeutically effective amount” or “dose” as used herein means the amount of medicine needed to reduce the symptoms of a disease in a patient. The dose of medicine will be tailored to the individual requirements in each case. This dose can vary widely depending on numerous factors, such as the severity of the patient's illness, the age and general health of the patient, other drugs with which the patient is being treated, the method and form of administration of medicine, and the experience of the attending physician. Typically, treatment is started with a large initial “loading dose” to rapidly reduce or eliminate the virus and followed by tapering the dose to a level sufficient to prevent an outbreak of infection.

The term “patient” means a mammal including but not limited to humans, cattle, pigs, sheep, chickens, turkeys, buffaloes, llamas, ostriches, dogs, cats, hamsters, and mice, preferably the patient is a human.

The term “active ingredient (substance)” as used herein means aprotinin and an inhibitor of RNA viruses used in a pharmaceutical composition or drug.

### SUMMARY

In one aspect, the present application relates to pharmaceutical combination therapy and prevention of SARS-CoV-2 and/or disease associated with this infection, including COVID-19, using aprotinin (APR), favipiravir (FVP) and/or molnupiravir (MOV) and excipients.

In another aspect, the present application provides a pharmaceutical kit for a pharmaceutical combination therapy and prevention of SARS-CoV-2 and/or disease associated with this infection, including COVID-19, consisting of: the parenteral drug Trasylol®, or Gordox®, or Aprotex®, or Antagosan®, or Contrycal®, or Traskolan®, or others parenteral drugs including APR, or aqueous or saline solution containing APR and a drug including favipiravir or aqueous or saline solution containing FVP, or its lyophilizate and/or a drug including MPV or aqueous or saline solution containing MPV or its lyophilizate.

In another aspect, the present application provides a pharmaceutical composition in the form of an aqueous solution (APC) or lyophilizate (PCL) for a pharmaceutical combination therapy and prevention of SARS-CoV-2 and/or disease associated with this infection, including COVID-19, including APR, FVP and/or MPV and excipients.

In some embodiments, the excipients are selected from the series: water, sodium chloride, L-lysine monohydrate, 2-hydroxy-beta-cyclodextrin, betadex sulfobutyl ether sodium, sodium hydroxide, hydrochloric acid, benzyl alcohol, ethanol, glycerin, dimethyl sulfoxide, peppermint oil, 1,1,1,2-tetrafluoroethane, and others.

APR is an inhibitor of the natural proteases with a long history of clinical use since the 1960s and a good safety profile. APR, under the trade names Trasylol®, or Gordox®, or Aprotex®, or Antagosan®, or Contrycal®, or Traskolan®, and others, is used as an intravenous medication given by injection to reduce bleeding during complex surgeries such as heart and liver surgery, as an antiviral drug for the treatment and prevention of viral respiratory diseases (U.S. Pat. No. 5,723,439).

Parenteral drugs Trasylol®, Gordox®, Aprotex®, Antagosan®, Contrycal®, Traskolan®, and others are aqueous solutions of APR with an APR activity of 5000-10000 KIU/ml containing excipients selected from the series: sodium chloride, sodium hydroxide, 1M hydrochloric acid solution, benzyl alcohol, and others. These parenteral drugs are used to prevent intraoperative blood loss and reduce the volume of blood transfusion in liver and heart transplants, coronary artery bypass grafting using a heart-lung machine in adult patients who are at increased risk of bleeding or need blood transfusion. These drugs are also recommended as a preventive treatment for patients who are likely to be at an increased risk of bleeding or need transfusion.

APR inhibits the entry of SARS-CoV-2 into cells ((a) M. Hoffmann et al. SARS-CoV-2 Cell Entry Depends on ACE2 and TMPRSS2 and Is Blocked by a Clinically Proven Protease Inhibitor. Cell 2020, 181 (2), 271-280. e8. doi: 10.1016/j.cell.2020.02.052; (b) D. Bojkova et al. Aprotinin Inhibitors SARS-CoV-2 Replication. Cells 2020, 9 (11), 2377, doi: 10.3390/cells9112377.) and can be used for the prevention and treatment of SARS-CoV-2/COVID-19 (RU 2738885).

MPV (Lagevrio®, MK-4482, EIDD-2801) is an experimental oral anti-RNA viral drug, including influenza, Ebola, chikungunya and various coronaviruses. In 2021, an international application (WO/2021/159044) was filed for a pharmaceutical composition including, inter alia, MPV for the treatment of SARS-CoV-2.

FVP sold under the brand names Avigan®, Avifavir Areplivir® (tablets and lyophilizate), Coronavir®, and other analogues, is an antiviral medication used to treat influenza in Japan. It is also being studied to treat a number of other viral infections, including SARS-CoV-2 (Du YX, Chen XP. Favipiravir: pharmacokinetics and concerns about clinical trials for 2019-nCoV infection. Clinical Pharmacology and Therapeutics 2020, 108 (2), 242-247. doi:10.1002/cpt.1844). FVP is a prodrug that is metabolized to its active form, favipiravir-ribofuranosyl-5′-triphosphate (favipiravir-RTP), available in both oral and intravenous formulations (Guedj J. et al. (March 2018). Antiviral efficacy of favipiravir against Ebola virus: A translational study in cynomolgus macaques. PLOS Med. 2018, 5(3), e1002535; doi:10.1371/journal.pmed.1002535. Smee D. F. et. al. Intracellular metabolism of favipiravir (T-705) in uninfected and influenza A (H5N1) virus-infected cells. J. Antimicrob. Chemother. 2009, 64(4), 741-746; doi:10.1093/jac/dkp274.).

The authors of the present application have unexpectedly found that the active ingredients (APR, FVP and/or MPV) included in the new anti-SARS-CoV-2/COVID-19 viral pharmaceutical composition do not interact with each other during the preparation, short-term storage, and use of the composition as a parenteral drug.

Another aspect of the present application is directed to anti-SARS-CoV-2 viral disease and/or disease associated with this infection, including COVID-19, parenteral drug, which is APC of this invention.

The parenteral drug and APC according to this invention is intended for the prevention and treatment mainly of SARS-CoV-2 and COVID-19.

Another aspect of the present application is directed to the containing APC of this invention container.

Another aspect of the present application is directed to an ampoule and a bottle containing APC of this invention.

Another aspect of the present application is directed to an inhaler (nebulizer) selected from a number of compressor or ultrasonic or electronic mesh inhaler (nebulizer) containing APC of this invention.

Another aspect of the application is directed to a pocket or portable inhaler (nebulizer) containing APC of this invention.

Another aspect of the application is directed to a container, which in some embodiments is a can for nasal spray containing APC of this invention.

Another aspect of the application is directed to a method for the prevention and/or treatment of SARS-CoV-2 infection and/or disease associated with this infection in a patient by parenteral (intravenous, inhalation, or intranasal) administration to the patient of a saline solution APR and oral FVP and/or MOV and in a therapeutically effective amount once or twice a day (as prescribed by a physician, depending on the patient's condition).

Another aspect of the application is directed to the use of the parenteral saline solution APR and oral FVP and/or MOV in a therapeutically effective amount once or twice a day (as prescribed by a physician, depending on the patient's condition) for the prevention and/or treatment of SARS-CoV-2 infection and/or disease associated with this infection, including COVID-1.

Another aspect of the application is directed to the use of the parenteral saline solutions APR, FVP and/or MPV in a therapeutically effective amount once or twice a day (as prescribed by a physician, depending on the patient's condition) for the prevention and/or treatment of SARS-CoV-2 infection and/or disease associated with this infection, including COVID-1. Instead of saline solution of APR can be used Trasylol®, or Gordox®, or Aprotex®, or Antagosan®, or Contrycal®, or Traskolan®, or others are aqueous solutions of APR.

Another aspect of the present application is directed to a method for the prevention and/or treatment of SARS-CoV-2 infection and/or disease associated with this infection in a patient by a parenteral (intravenous, inhalation, or intranasal) administration to the patient of APC of the present invention in a therapeutically effective amount once or twice a day (as prescribed by a physician, depending on the patient's condition).

In some embodiments of the present application, prophylaxis of patients is carried out with a therapeutically effective amount of a nasal spray, which is APC of this invention.

In some embodiments of the present application, prophylaxis is carried out using nasal spray cans in the nose (nasopharynx) and throat 3-6 times a day in each nostril and throat with a therapeutically effective amount of APC according to the present invention.

In some embodiments of the present application, the prophylaxis and treatment of patients is carried out by inhalation using ultrasonic nebulizers 3-6 times a day for 5-7 days a therapeutically effective amount of APC according to this invention.

Another aspect of the present application is directed to the use of the APC or the parenteral drug of the present invention for the prevention and/or treatment of SARS-CoV-2 and/or disease associated with this infection by intravenous, or inhalation, or intranasal administration to a patient of the APC or the parenteral drug of the present invention in a therapeutically effective amount.

Another aspect of the present application is directed to the use of the APC or the parenteral drug of the present invention in a therapeutically effective amount for the prevention and/or treatment of SARS-CoV-2 and/or disease associated with this infection, including COVID-19.

The use of the new a pharmaceutical kit and APC and the new parenteral drug of the present application in pharmaceutical combination therapy and prevention of SARS-CoV-2 and/or disease associated with this, including COVID-19, was more effective than monotherapy with FVP and/or MPV.

On the one hand, when using APC or the separate saline solutions APR, FVP and/or MPV, additionally significantly simplifies the process of prevention and treatment of patients compared with separate combination therapy by the oral drug+the parenteral drug and allows treatment of patients unable to take oral drugs.

In addition, the use of the pharmaceutical combination therapy of the present application is more effective against SARS-CoV-2, because it uses drugs with two different mechanisms of action. APR is a nonspecific inhibitor of the serine proteases—especially trypsin, chymotrypsin, plasmin, kallikrein, and an inhibitor of entry of SARS-CoV-2 into the cell. FVP and/or MPV are inhibitors of a replication of SARS-CoV-2 in cells.

In addition, the use of the pharmaceutical combination therapy of the present application provides through the use of APR effective inhibition of inflammation and thrombosis in the moderate and severe COVID-19 patients.

Another aspect of the present application is directed to a method of obtaining the APC or the parenteral drug of the present invention by dissolving APR, FVP and/or MOV, and excipients in saline (0.9% aqueous sodium chloride solution).

Another aspect of the present application is directed to a method of obtaining APC or the new parenteral drug of the present invention by dissolving a lyophilizate containing APR, FVP and/or MPV and excipients in saline.

The active ingredients and the PCL of the present application retain their activity in a convenient lyophilized form for storage.

Another aspect of the present application is directed to the use of APR in the form of a powder or a lyophilizate, or a concentrate, or a drug selected from the group consisting of Trasylol®, or Gordox®, or Aprotex®, or Antagosan®, or Contrycal®, or Traskolan®, and others for preparing the APC or the parenteral drug of the present invention.

Another aspect of the present application is directed to the use of FVP and/or MOV in the form of a powder, or a lyophilizate, or a concentrate for preparing the APC or the parenteral drug of the present invention.

Another aspect of the present application is directed to the use of FVP and/or MPV in the form of a powder, or a lyophilizate, or a concentrate and APR as Trasylol®, or Gordox®, or Aprotex®, or Antagosan®, or Contrycal®, or Traskolan®, and others for preparing the APC or the parenteral drug of the present invention.

Another aspect of the present application provides a method for preparing the PCL by dissolving APR, FVP and/or MPV in the form of a powder, or a lyophilizate, or the water solution, and excipients in water or saline followed by lyophilization of the resulting mixture.

Another aspect of the present application a method for preparing the PCL by dissolving FVP and/or MOV in the form of a powder, or a lyophilizate, or the water solution, and excipients in Trasylol®, or Gordox®, or Aprotex®, or Antagosan®, or Contrycal®, or Traskolan®, and others followed by lyophilization of the resulting mixture.

The APC can be obtained, including immediately before use, by sequential dissolution in physiological solution of the crystalline APR or its lyophilizate, the crystalline FVP and/or MOV or their lyophilizate and, if necessary, excipients.

The APC can be obtained, including immediately before use, by dissolving the crystalline FVP and/or MOV or their lyophilizate in an aqueous solution of APR or in known drugs that are aqueous solutions of APR, for example, Trasylol®, or Gordox®, or Aprotex®, or Antagosan®, or Contrycal®, or Traskolan®, and others, and, if necessary, bringing the resulting compositions to the required concentration of active ingredients with saline.

The new APC can be obtained, including immediately before use, by sequential dissolution in saline of crystalline APR or its lyophilizate, for example, Contrykal®, the crystalline FVP and/or MPV or their lyophilizate and, if necessary, excipients.

During intraperitoneal treating with LPC the transgenic mice (B6.Cg-Tg(K18-ACE2)2Prlmn/HEMI Hemizygous for Tg(K18-CE2)2Prlmn from Jackson Immunoresearch, West Grove, PA, USA; females, age—6-8 weeks, weighing 19-24 g) infected with mouse-adapted SARS-CoV-2 (“Dubrovka” strain, identification number GenBank: MW161041.1) a statistically significant reduction in virus titer by 4.2-4.6 orders of magnitude was obtained in the lungs of infected animals, compared with the control group of infected but untreated animals.

During intravenous treating with LPC the Syrian hamsters weighing 100-120 g (State Scientific Center for Virology and Biotechnology “Vector” of Rospotrebnadzor, Russia) infected with SARS-CoV-2 (strain hCoV-19/Australia/VIC01/2020), a statistically significant reduction in virus titer by order of magnitude was obtained in the lungs of infected animals, compared with the control group of infected but untreated animals.

Below are examples of the preparation and use of an anti-RNA viral preparation (anti-RNA viral pharmaceutical composition), confirming but not limiting the scope of the present application.

Example 1. Preparation of the PPC's comprising APR and MOV

PPC 1. MOV from Jiangsu Zenji Pharmaceuticals Ltd., China (800.0 mg), and APR from Wanhua Biochem, China (92.4 mg, ˜500,000 KIU), with an activity of 5400 KIU mg were dissolved in saline (50 ml) under ultrasonic stirring for 5 minutes to yield PPC 1 containing MOV (16 mg/ml) and APR (10000 KIU/ml).

PPC 2. Saline (9 ml) was added under ultrasonic stirring to PPC 1 (1 ml) to yield 10 ml of PPC 2 containing MOV (1.6 mg/ml) and APR (1000 KIU/ml).

PPC 3. Saline (999 ml) was added under ultrasonic stirring to PPC 1 (1 ml) to yield 1000 ml of composition PPC 3 containing MOV (16.0 μg/ml) and APR (10 KIU/ml).

PPC 4. Dissolve 800.0 mg MOV from Jiangsu Zenji Pharmaceuticals Ltd. (China) and 92.4 mg (˜500,000 KIU) APR from Wanhua Biochem (China) with an activity of 5400 KIU/mg in 50 ml of saline under ultrasonic stirring for 5 minutes to yield pharmaceutical PPC 4 containing 16 mg/ml MOV and 10000 KIU/ml APR.

PPC 5. MOV from Jiangsu Zenji Pharmaceuticals Ltd, China (30.0 mg) and APR from Wanhua Biochem, China (37.0 mg, 200,000 KIU), with an activity of 5400 KIU/mg were dissolved in saline (20 ml) under ultrasonic stirring for 5 minutes to yield PPC 5 containing MOV (1.0 mg/ml) and APR (10,000 KIU/ml).

PPC 6. 50.0 mg of MOV from Jiangsu Zenji Pharmaceuticals Ltd, China were dissolved in 10 ml Gordox® under ultrasonic stirring for 5 minutes to yield PPC 6a containing 5.0 mg/ml of MOV and 10000 KIU/ml of APR. PPC 6a obtained as an aqueous solution was frozen and lyophilized. Received PPC 6b in the form of a lyophilizate containing 50 mg of MOV and 100,000 KIU of APR. 100.0 mg of MOV from Jiangsu Zenji Pharmaceuticals Ltd, China were dissolved in 10 ml Gordox® under ultrasonic stirring for 5 minutes to yield PPC 7a containing 10.0 mg/ml of MOV and 10000 KIU/ml of APR.

PPC 7. 100.0 mg of MOV from Jiangsu Zenji Pharmaceuticals Ltd, China were dissolved in 10 ml Gordox® under ultrasonic stirring for 5 minutes to yield PPC 7a containing 10.0 mg/ml of MOV and 10000 KIU/ml of APR. PPC 7a obtained as an aqueous solution was frozen and lyophilized. Received PPC 7b in the form of a lyophilizate containing 50 mg of MOV and 100,000 KIU of APR.

PPC 8. 400.0 mg of MOV from Jiangsu Zenji Pharmaceuticals Ltd, China were dissolved in 20 ml Gordox® under ultrasonic stirring for 5 minutes to yield PPC 8 containing 20.0 mg/ml of MOV and 10000 KIU/ml of APR.

Example 2. Stability of PPC 3. The stability of PPC 3 was studied by UV spectroscopy on an Agilent 8453 spectrophotometer after storage under normal conditions and under stress tests (Table. 1).

The optical densities of the maxima of the initial spectra (condition 1) PPC 3 differ greatly from those in the stress test. The percentage of change in optical density (A) under conditions 3-5 compared to optical density under Conditions 1 is >2% (Table 1). This indicates that PPC 3 and other PPC's from Example 1 are limitedly stable under rapid tests conditions and them must be used within a few hours after preparation.

TABLE 1
Optical density at the maxima of absorption bands in the UV spectra of PPC
3 immediately after solution preparation (Condition 1) and after exposure for 24 hours in the light at
25° C. (2), after exposure for 48 hours in the dark at 3-5° C. (3), after exposure for 48 hours in the dark
at 25° C. (4), and after exposure for 48 hours in the dark at 60° C. (5). Δ, % is the percentage of change
in optical density under conditions 2-5 compared to optical density under Conditions 1.

| | Maxima in UV spectra and percent change in absorbance (Δ) | | | |
|---|---|---|---|---|
| | under Conditions 2-5 compared to absorbance under Conditions 1 | | | |
| | 235 nm | | 275 nm | |
| Conditions | Optical density | Δ, % | Optical density | Δ, % |
| 1 | 0.68660 | — | 0.33161 | — |
| 2 | 0.68450 | −0.31 | 0.33547 | 1.16% |
| 3 | 0.70550 | −2.75 | 0.34651 | 4.49% |
| 4 | 0.70322 | −2.42 | 0.34737 | 4.75% |
| 5 | 0.64800 | −5.62 | 0.33297 | 0.41% |

Example 3. A device for inhalation therapy and prevention of SARS-CoV-2/COVID-19. 5-10 ml of PPC 2 containing MOV (1.6 mg/ml) and APR (1000 KIU/ml) according to Example 1, is placed into a compression nebulizer Omron NE-C300 Complete or in a portable ultrasonic Feellife Aerogo mesh nebulizer and is receive the device for inhalation therapy and prevention of SARS-CoV-2/COVID-19.

Example 4. Devices for nasal spray therapy and prevention of SARS-CoV-2/COVID-19. 5-10 ml of PPC 2 containing MOV (1.6 mg/ml) and APR (1000 KIU/ml) according to Example 1, is placed into a plastic can for nasal and is receive the device for nasal therapy and prevention of SARS-CoV-2/COVID-19.

Example 5. Treatment with PPC of transgenic mice infected with mouse-adapted SARS-CoV-2.

In the experiment, 5 groups of transgenic mice (B6.Cg-Tg(K18-ACE2)2Prlmn/HEMI Hemizygous for Tg(K18-CE2)2Prlmn from Jackson Immuno-research, West Grove, PA, USA), females, age—6-8 weeks, weighing 19-24 g, were formed, 4 animals per group.

Group 1—control group, untreated mice: on day 1 in the morning the mice were infected with SARS-CoV-2, and then 5 ml/kg of water for injection was intragastrical administered immediately after infection and in the evening of the same day.

Group 2—treatment with Gordox®—10 000 KIU/ml of APR. Dose: 50 000 KIU/kg APR.

Group 3—treatment with saline solution of MOV—5 mg/ml. Dose: 25.0 mg/kg of MOV.

Group 4—treatment with the PPC 6 from Example 1. Dose: 50 000 KIU/kg of APR+25 mg/kg of MOV.

Group 5—treatment with the PPC 7 from Example 1. Dose: 50 000 KIU/kg of APR+50 mg/kg of MOV.

Treatment regimen for transgenic mice: parenteral (intraperitoneal) administration of drugs 2 times a day; day 0-1 hour before infected with mouse-adapted SARS-CoV-2 (“Dubrovka” strain, identification number GenBank: MW161041.1) and 6-8 hours after infection; days 1, 2, 3-2 times a day, for a total of 4 days (days 0, 1, 2, 3); Day 4—lung sampling from all animals to assess the virus titer in the lungs, visual assessment of the lungs and transfer of the lungs for histology; days 0-4—daily assessment of body weight and condition of mice.

On day 0, animals from all groups were infected with the SARS-CoV-2 “Dubrovka” virus (10^{3.5} TCID₅₀/ml). All mice were infected intranasally under light ether anesthesia in a volume of 30 μl for both nostrils.

Euthanasia (painless killing of the animal) was carried out by the responsible person in accordance with the existing ethical requirements, by dislocation of the cervical vertebrae with preliminary anesthesia with ether. Euthanasia was performed promptly after the end of the experiments.

On day 4 post-infection with the virus, the animals in each group were sacrificed and the lungs were removed under sterile conditions. One lung was fixed in formalin for further histology, the second lung was prepared to measure the virus titer. To do this, after washing three times in a solution of 0.01 M phosphate buffered saline (PBS), the lungs were homogenized and resuspended in 1 ml of cold sterile PBS. The suspension was cleared from cell debris by centrifugation at 2000 g for 10 min, and the supernatant was used to determine the infectious titer of the virus in cell culture and to perform PCR. The obtained samples were stored at −80° C. until the experiments were carried out.

To determine the infectious titer of the virus from the lungs of mice, Vero CCL81 cells were seeded in 96-well Costar plates with an average density of 20,000 cells per well and grown in DMEM medium in the presence of 5% fetal calf serum, 10 mM glutamine and antibiotics (penicillin 100 IU/well). ml and streptomycin 100 μg/ml) until a complete monolayer is formed (within 3 days). Before infection with the virus, the cell culture was washed twice with DMEM medium without serum. 10-fold dilutions of each lung virus sample were prepared from 10-1 to 10-7. The prepared dilutions in a volume of 200 μL were added to cell culture plates and incubated in 5% CO2 at 37° C. for 5 days until a cytopathic effect (CPE) appeared in virus control cells. Accounting for the result of the manifestation of CPP in cells was carried out using a quantitative MTT test. The virus titer was calculated using the Ramakrishnan M. A formula in the Excel program [M. A. Ramakrishnan. Determination of 50% endpoint titer using a simple formula. World J. Virol. 2016, 5, 85-86. doi: 10.5501/wjv.v5.i2.85] and expressed as lgTCID₅₀/ml (TCID₅₀—The median tissue culture infectious dose is defined as the dilution of a virus required to infect 50% of a given cell culture.) [I. Leneva et al. Antiviral Activity of Umifenovir In Vitro against a Broad Spectrum of Coronaviruses, Including the Novel SARS-CoV-2 Virus. Viruses 2021, 13(8): 1665. doi: 10.3390/v13081665]. Next, the average titer value for samples from mice of the same group was calculated.

The effectiveness of the drugs in a model of the transgenic mice infected with mouse-adapted SARS-CoV-2 was assessed according to decrease in the titer of the virus in the lungs of animals after 4 days.

The obtained digital data were subjected to statistical processing in the “Statistica 8.0” software. The results are shown in Table 2.

TABLE 2
The effectiveness of the drugs and control (untreated mice) on the model the
transgenic mice infected with mouse-adapted SARS-CoV-2.

| Group | Dose of drug | logTCID₅₀/ml | ^{b}Decrease in logTCID50/mL |
|---|---|---|---|
| 1 | Control | 8, 83; 9, 50; 8, 17; 8, 5; 8, 83 (8, 77) | — |
| 2 | ªAPR | 7, 17; 6, 50; 8, 17; 7, 17 (7, 25) | 1.52 |
| 3 | 25 mg/kg MOV | 5, 83; 3, 83; 5, 50; 4, 83 (5, 0) | 3.77 |
| 4 | ªAPR + 25 mg/kg MOV | 4, 83; 5, 50; 3, 50; 3, 83; 5, 17 (4, 57) | 4.20 |
| 5 | ^{a}APR + 50 mg/kg MOV | 3, 83; 4, 83; 4, 50; 3, 50 (4, 16) | 4.61 |

^{a}50000 KIU/kg APR.
^{b}% reduction in virus titer (logTCID₅₀/ml) compared to control (group 1).

As can be seen from Table 2, parenteral (intraperitoneal) treatment of mice infected with SARS-CoV-2 resulted in a reduction in virus titer in the lungs of infected animals compared to a control group of infected but untreated animals. Thus, with parenteral monotherapy with APR or MOV, a decrease in virus titer (log TCID₅₀/ml) by 1.52 and 3.77, respectively, was observed. At the same time, combination parenteral therapy of APR+MOV provides a decrease in virus titer by 4.2 and 4.6 orders of magnitude respectively for groups 4 and 5.

Example 6. Intravenous treatment of Syrian hamsters infected with the SARS-CoV-2 using the PPC 8 from Example 1 containing MOV and APR.

The efficacy of the PPC 8 of the present invention was evaluated using a model of SARS-CoV-2 infection in Syrian hamsters [R. Boudewijns et al. STAT2 signaling as double-edged sword restricting viral dissemination but driving severe pneumonia in SARS-CoV-2 infected hamsters. BioRxiv preprint. doi: 10.1101/2020.04.23.056838].

The strain SARS-CoV-2 hCoV-19/Australia/VIC01/2020 was obtained from the State Research Center of Virology and Biotechnology VECTOR (Russia). The infectious virus was isolated by sequential passage in Vero E6 cells. The titer of the viral suspension was determined by endpoint dilution on Vero E6 cells using the Reed-Muench method. The work related to the live virus was carried out under isolated laboratory conditions that meet the international BSL-3+VECTOR requirements.

Vero E6 cells from VECTOR's Collection of Cell Cultures were cultured in Minimum Essential Medium (MEM) (Gibco) supplemented with 10% fetal bovine serum (Integro), 1% L-glutamine (Gibco), and 1% Bicarbonate (Gibco). Endpoint titrations were performed with a medium containing 2% fetal bovine serum.

Wild-type Syrian hamsters at the age of 6-10 months weighing 100-120 g from State Scientific Center for Virology and Biotechnology “Vector” of Rospotrebnadzor (Russia) were kept with unlimited access to food and water. Hamsters were randomized into 4 cohorts, 8 animals in each cohort (4 males and 4 females).

Hamsters were anesthetized with zoletil-xyla and inoculated into each nostril with 50 μl anesthetic combination containing 10³ TCID₅₀.

Group 1—control group, untreated hamsters. Dose: 5 ml/kg saline.

Group 2—treatment with Gordox®—10 000 KIU/ml of APR. Dose: 10000 KIU/kg APR.

Group 3—treatment with saline solution of MOV—20 mg/ml. Dose: 100.0 mg/kg of MOV.

Group 4—treatment with the PPC 8 (MOV—20 mg/ml+10000 KIU/ml of APR) from Example 1. Dose: 50 000 KIU/kg of APR+100 mg/kg of MOV.

The drugs were injected under light isoflurane anesthesia intravenously, 2 times a day for 4 days, starting the first injection one hour before infection, 6 hours after infection, then for 3 days after 12 hours.

Hamsters were checked daily for appearance, behavior and weight. On the 4th day after infection, the hamsters were euthanized by intravenous injection of 500 μl doletal (200 mg/ml sodium pentobarbital, Vétoquinol SA). Hamster lung tissues were harvested after sacrifice and homogenized using a Precellys homogenizer in a 350 μl RNeasy lysis buffer (RNeasy Mini kit, Qiagen) and centrifuged (10,000 rpm, 5 min) to remove cell debris. RNA was extracted according to the manufacturer's instructions. Real-time PCR was performed on the LightCycler96 platform (Roche) using the iTaq Universal Probes One-Step RT-qPCR kit (BioRad) [R. Boudewijns et al. STAT2 signaling as double-edged sword restricting viral dissemination but driving severe pneumonia in SARS-CoV-2 infected hamsters. BioRxiv preprint. doi: 10.1101/2020.04.23.056838].

For histological analysis, lung tissue was fixed in 4% formaldehyde, embedded in paraffin, and stained with hematoxylin-eosin. Damage was assessed on a scale from 1 to 3: stagnation, intraalveolar bleeding, apoptotic bodies in the bronchial epithelium, necrotic bronchiolitis, perivascular edema, bronchopneumonia, perivascular inflammation, peribronchial inflammation, and vascular inflammation.

Statistical analysis was performed using the GraphPed Prism software from GraphPed Software, Inc. Statistical significance was determined using the Mann-Whitney nonparametric U-test. The values of P≤0.05 were considered significant.

An analysis of the results obtained showed that PPC 8 comprising MOV and APR demonstrated a high anti-SARS-CoV-2/COVID-19 efficacy. Thus, in comparison with the control group, after intravenous treatment of Syrian hamsters infected with the SARS-CoV-2 of drug PPC 8, the titer of SARS CoV-2 in the tissues of the lungs decreased by more than an order of magnitude compared to the control group.

1. A method for treating a SARS-CoV-2 infection or a disease associated with a SARS-CoV-2 infection, comprising:
forming a solution comprising a therapeutically effective amount of aprotinin and Molnupiravir, wherein the solution exhibits less than 2% change in optical density at 25° C. within 24 hours of preparing the solution, and
administering to a patient the solution.

2. (canceled)

3. The method of claim 1,
wherein the disease associated with SARS-CoV-2 is COVID-19.

4. The method of claim 1,
wherein aprotinin is comprised in a pharmaceutical composition containing dissolved aprotinin or a finished dosage form.

5. The method of claim 1,
wherein molnupiravir is in a lyophilizate form or a finished dose form containing molnupiravir.

6. (canceled)

7. The method of claim 1, comprising:
administering parenterally to a mammal a therapeutically effective amount of aprotinin and molnupiravir.

8. (canceled)

9. The method of claim 7,
wherein the parenteral administration is intravenous, inhalation, or nasal administration.

10. (canceled)

11. A pharmaceutical composition for the treatment of a SARS-CoV-2 infection or a disease associated with SARS-CoV-2 infection, comprising:
aprotinin and molnupiravir in therapeutically effective amounts, and excipient.

12. (canceled)

13. The pharmaceutical composition of claim 11,
further comprising a pharmaceutically acceptable excipient selected from the group consisting of water, sodium chloride, L-lysine monohydrate, 2-hydroxy-beta-cyclodextrin, betadex sulfobutyl ether sodium, sodium hydroxide, hydrochloric acid, benzyl alcohol, ethanol, glycerin, dimethyl sulfoxide, peppermint oil, and 1,1,1,2-tetrafluoroethane.

14-17. (canceled)

18. The method of claim 1,
wherein aprotinin and Molnupiravir are administered in a ratio of 16.0 μg/ml of Molnupiravir per 10 KIU/ml of aprotinin.
